(19) United States
(12) Patent Application Publication
(10) Pub. No.: US 2003/0161799 A1
(43) Pub. Date: Aug. 28, 2003

(54) Practical and humane method for armpit odor care

(76) Inventors: Georgiy Borisovich Kuznetsov, Columbus, OH (US); Marina Mikhailovna Komissarova, Columbus, OH (US)

Correspondence Address: GEORGIY KUZNETSOV, 2714 WINDHAM CLUB, COLUMBUS, OH, 43219, US

(21) Appl. No.: 10/083,307

(22) Filed: Feb. 26, 2002

(51) Int. Cl. A61K 7/32; A61K 31/496; A61K 31/4196; A61K 31/4178

(52) U.S. Cl. 424/65; 514/254.07; 514/397; 514/383

(57) ABSTRACT

A set of methods presented for preventing and curing axillary malodors through treating the skin, and fomites with safe, non-restricted, widely available agents.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE OR A COMPUTER PROGRAM LISTING COMPACT DISK APPENDIX

Not Applicable.

### BACKGROUND OF THE INVENTION

This invention relates to personal care, specifically to substances and methods for managing human axillary malodors.

Axillary malodors are believed to be produced by bacteria digesting certain components of human sweat. Although there are many species involved, the worst smelling part is attributed to the group loosely identified as coryneforms, or Corynebacteria.

Axillary malodor normally emerges in puberty, as apocrine glands secretion starts in the skin. It haunts most of the adult population, often getting worse with age. Some children develop axillary malodor unusually early. Foul body odors promote aggressive behavior and social tension. There are people producing malodor so strong that the others cannot tolerate them.

At present dominating art of axillary malodor care comprises antiperspirants and deodorants. Deodorants destroy (or mask) the odor itself. Some of them contain mild antiseptics like triclosan, able to diminish bacterial population. Antiperspirants block sweating. This seems to be not a right idea to many. Rumors blame antiperspirants for skin and breast cancer. Medical sources deny this, but admit connection between antiperspirants and “harmless” skin lumps. Antiperspirants can also produce various skin irritations.

There also are “non-traditional” products on the market, like mineral salts of undisclosed composition claimed not to be antiperspirants—although antiperspirants are salts. Antiperspirant pills currently advertised on the Internet may comprise another example.

All antiperspirant and deodorant-type solutions are intended for everyday use. The protection they provide lasts for up to 48 hours.

Bacteria producing axillary malodors are currently driving 4 billion dollars market of personal care products. Scientists call them normal, non-pathogenic or even commensal organisms. It is well known, however, that they can be treated like a skin infections. For this purpose, dermatologists routinely prescribe antibiotics, most commonly erythromycin in topical forms. Axillary malodor treatment also occurs as a side effect of some antibiotics and sulfa drugs administered internally. In both cases, infection recovers soon.

Coryneform bacteria do not make spores, and do not move on their own. They attach themselves to the skin, and a colony spreads as its members multiply, unless an external force helps them to out-migrate.

In armpits, coryneforms may colonize sweat ducts. This is why axillary malodor seems to belong to the skin itself. Besides these general considerations we could not find any knowledge specific to Corynebacteria found to armpit, as to do they live anywhere else on human bodies, how does this infection survive outside of the hosts, how does it spread, and how to cure it.

Iodine tincture is a little known home remedy. Rubbing alcohol is another one. Oxides of certain metals, most notably titanium and zinc, are powerful axillary malodor stoppers. The latter are widely used in baby diaper creams and sunscreens.

Some people report suppressing axillary malodor using over-the-counter antibiotic creams (contains neomycin and polymyxin B, may also include gramicidin or bacitracin).

Social awareness of axillary malodor problem is poor. People having perfectly normal condition often think they are unlucky ones among the majority who do not smell this bad. Asians suffer the worst, because most of them are not susceptible to this infection. Through our web site killbo.com, we met Chinese girl who was seriously thinking about suicide. She and her mother were the only people having this problem she new. Like many Asians, she ended up with a surgery.

Summing up, the disadvantages of the prior arts are:

(a) Using antiperspirants and deodorants we cover our skin on a daily basis with extraneous substances those are unpleasant at least, not to mention giving up our natural right to sweat. If “mild” bactericidal substances are involved, it is questionable with regards to developing antimicrobial resistance.

(b) Exterminating bacteria producing axillary malodor requires prescription-only medication, and do not ensure long-lasting relief.

(c) Non-prescription remedies like iodine and alcohol are also painful, anesthetic, and may have adverse effect to the skin.

(d) The goal of axillary malodor science and technology seems to be selling the products, not solving the problem. Amazingly little is known about the infection causing this phenomenon.

### BRIEF SUMMARY OF THE INVENTION

In accordance with the present invention, the skin and fomites are occasionally treated with non-restricted, affordable, widely available and practical agents ensuring long-lasting extermination of axillary malodors.

Several objects and advantages of the present invention are:

(a) To provide a method for exterminating the infection producing axillary malodor on the skin using safe, inexpensive, practical, non-prescription substances.

(b) To provide a method for exterminating axillary malodor production in fomites, for example, in articles of underwear contacting armpits, as well as for preventing the infection from returning from fomites to the host, and from spreading to other people.

(c) To provide a method for curing axillary malodor-producing infection through exterminating it in fomites, and on the skin at the same time.

Once a mature infection has been cured, the person typically stays free from axillary malodor for months. Further occurrences of infection are much easier to intercept, provided they are recognized and treated in time.

### BRIEF DESCRIPTION OF THE SEVERAL VIEWS OF THE DRAWING

Not Applicable.

### DETAILED DESCRIPTION OF THE INVENTION

A. Method for Treating the Skin

We have found that axillary malodor can be successfully exterminated through topical application of substances collectively called azoles, or imidazoles.

Azoles are known and sold as antifungal medicines. They are supposed to deplete ergosterol in fungal cells membranes. This does not necessarily imply that bacterial theory of axillary malodors is incorrect. The fact that azoles are effective against at least some Corynebacteria is not completely unknown, although this use is not advertised or recommended by drug manufacturers. For example, on a few occasions we found clotrimazole mentioned in medical literature as a good remedy for pitted keratolysis and erythrasma.

With regards to the present invention, erythrasma may be particularly significant. Erythrasma is a skin disease caused by Corynebacterium minutissimum. As the name suggests this bacterium is relative to those blamed for axillary malodor.

In case of erythrasma, the colony appears as slowly growing, pink or brown patch, often originating in armpits. The official medicine is, of course, erythromycin, although, according to medical practitioners and associations, azoles provide a robust non-prescription alternative treatment.

Non-prescription azoles sold in groceries are intended for treating feet or vaginal fungal infections. Essentially the same compositions are often sold for feet and vaginal applications under different labels and in different forms. For axillary use, we strongly prefer them as creams.

The active ingredients found in over-the-counter antifungal creams as of now are clotrimazole, miconazole, tioconazole, butoconazole, econazole, terconazole, ketoconazole, and fenticonazole (this list is not all-inclusive, and we wish not to be bound by it). Terbinafine and tolnaftate have showed some effect too.

The most widely used, and, probably, the safest, albeit not the most efficient of all are clotrimazole and miconazole. The latter is, in fact, miconazole nitrate. Some other medicines mentioned in this disclosure may appear as derivative substances also.

According to the drug information database accessible from the official website of National Institutes of Health, topical clotrimazole and miconazole are now considered harmless even for children, and women during the second and the third trimester of pregnancy.

Base composition of generic clotrimazole cream includes cetostearyl alcohol, cetyl esters wax, octyldodecanol, polysorbate, water, sorbitan monostearate and benzyl alcohol (1% as a preservative). This cream is colorless and odorless. It has a shelf life of up to 3 years at room temperature. The skin absorbs it seconds, so normally there are no fatty spots left on the garments.

Clotrimazole was the first azole approved for over-the-counter sale in the US in 1990. At present, there are more powerful alternatives. Some even require one-time application only.

B. Odor Changes Resulting from a Treatment

There are different bacteria species involved in axillary malodors production, and quite a number of their metabolites contribute to the bouquets. Although many of those substances are now identified, we prefer to describe the odor non-scientifically, because the invention we disclose here is, essentially, about self-help technique.

A typical mature axillary malodor is pungent, foul, durable, and resident. Two last attributes are particularly important.

We define axillary malodor as durable because it lasts, and fills the room. It is easy to tell that a shirt had been in use, even if this had happened many months ago.

We define axillary malodor as resident because it seems to go from inside the skin. It smells right after a shower.

Azole treatment changes armpit odor dramatically. Post-treatment odor is not resident: it removes with warm water. Washing armpits twice a day is enough to keep post-treatment odor at a socially acceptable level. Soaping armpits is not needed, and even not desirable.

Post-treatment odor is anaerobic. It develops only if armpit is closed tight. Being exposed to the air the post-treatment odor disappears in seconds, and does not fill the room. It is hard to tell a used shirt from a fresh one.

Post-treatment odor is not pungent. It is like those “natural” body scents appearing in modern deodorants. Different people smell different. Right and left armpit of the same person may have different smell too. There are summer, and winter odors. Post-treatment armpit odor reflects physical condition of the person, and even the things we drink or eat.

In our experiments, we encountered one case of axillary malodor not treatable with azoles. One of us probably has had it for years. After the first azole treatment there was slight, pungent smell left on the right side. It became strong after reinfecting from the underwear, and turned out to be not susceptible to azoles. Eventually it had been exterminated five months ago, and has not come back since then.

C. Broad-Spectrum Antibacterial Drugs

We further tried some other broad-spectrum non-prescription antimicrobial substances used against common skin infections. Undecylenic acid, salicylic acid, benzoyl peroxide, and even hydrogen peroxide were found to be functional against axillary malodors too.

Undecylenic acid is a natural component of human sweat. We chose it by analogy, because it is was the best known antifungal drug before azoles were brought to the market. As for salicylic acid and peroxides, they are widely used to contain acne, which is caused by another coryneform bacteria.

Undecylenic acid, salicylic acid and peroxides suffer from certain disadvantages. First of all, they all may cause pain, burning and irritations. This is particularly true for undecylenic acid. It is now available in 10% to 25% compositions those are too strong for sensitive armpit skin.

Concentrated undecylenic acid has distinctive unusual smell. Peroxides in all forms may discolor fabrics, and hair.

Another problem we encountered after using broad-spectrum drugs (including antibiotics) is that different, even stronger malodor may develop in a few days after their use.

Undecylenic acid, salicylic acid and peroxides apparently may be useful for treating malodors not susceptible to azoles. Nonetheless, we found them generally less practical. We believe that all broad-spectrum substances, including prescription drugs, better be avoided, unless they are really needed.

D. Method for Treating Fomites

We further found that repeated infection typically comes from the cloth contacted an infected armpit it the past. This phenomenon is obscured by the fact that the items of underwear are not equally contagious. Most of them appear to be harmless after washing with surfactants. Only a few shirts in a personal wardrobe may keep live bacterial culture.

Infected shirts can be slightly yellow or gray in armpits, and the odor of laundry detergent is stronger there. As a person free from axillary malodor puts such a shirt on, a typical axillary malodor appears on the cloth in approximately two hours, and stays until it washed. During wearing an infected shirt, a skin irritation may occur. Initial symptoms remove with warm water, but in a few days, axillary malodor emerges on the skin. It may take up to three weeks for it to mature.

In our experiments, we treated the armpits, then switched to all new underwear, and rigorously sterilized it with chlorine-based bleaches after every use. After living without axillary malodor for four to six weeks the tester returned to the old garments, not sterilized. The first five cases of recurring infection thus encountered were all traced back to such bad shirts.

We noticed that contaminated shirts are often something special to their owners, so they might be put on in anticipation of an emotionally stressful situation. It is believed that apocrine gland production increases in such moments.

Surfactants may not remove infection from the cloth. Chlorine and ammonium-based bleaches work very well, but are not good for many fabrics. Oxygen (peroxide) bleaches are gentler to cloths, but seem to be less efficient antiseptics too, so it is better to use them systematically.

Professional cleansing and disinfecting may be needed for fabrics other that cotton and lax, like wool, silk, synthetics, etc. Another approach is to wear such garments during the skin treatment, so they receive the medicine from armpits. In general, the substances effective against axillary malodor on the skin may be used to treat fomites vulnerable to crude sanitizing.

High temperature (for example, applied through dry ironing, or baking) may be used as a sanitizing agent too. It kills bacteria, but it does not clean residual substances from the cloth.

E. Method for Curing the Infection

As we disclosed above, infections causing axillary malodors extend from the skin to fomites, particularly to the underwear, towels, and bed linen. Depending on the personal habits, there may be other articles to consider—for example, axillary thermometers. The bacteria seem to be able to infest shirts, releasing malodor right to the outside world.

Although our methods for removing infection from the skin and fomites are of independent value, it is better to apply them together. If a method known prior to this invention is chosen to treat the skin, disinfecting fomites according to this invention is still beneficial since this provides long-lasting relief.

In other words, as axillary malodor is exterminated on the skin, articles contacting armpits have to be decontaminated and disinfected prior to using them again. Otherwise, recurring infections may happen often. If, and only if the infection is exterminated throughout its span we consider it cured for a given patient.

It makes sense to plan for disinfecting the household altogether, and, to that end, even to create a registry to keep track of people, things and actions taken.

However, if even a mild disinfection applies systematically to the underwear each time it is washed, and the recurring infections are treated as early as possible on the skin, the infection may as well be exterminated eventually.

F. Selecting the Medicine

To the best of our knowledge, all topical antifungal azoles are effective against axillary malodor, and may be used approximately as recommended for treating fungal infections. There are several factors to consider, however, including overall length of the treatment, how many times per day the medicine must be applied, and how it may affect the garment.

For example, clotrimazole treatment of mature axillary malodor takes two weeks. The cream has to be applied two times a day. The odor disappears typically between the fourth and the seventh day. Vaginal tioconazole is much more effective than clotrimazole, but it contains mineral oil staying on the skin.

All restrictions and reservations regarding the drugs are applicable to axillary malodor treatment. At present, we have no information on how antifungal creams may interact with antiperspirants and deodorants, so they better not to be used at the same time.

G. Handling Recurring Infections

There are two distinctive types of recurring infections. Axillary malodor may be contracted from underwear and other personal belonging, or (much less likely) from the outside. In both cases the sooner the infection is recognized, the easier it is to intercept. Fortunately living free from axillary malodor makes people more sensitive to it. An outbreak becomes something hard to miss.

If underwear smells after use, it's a good indication that the cloth is infected, so skin reinfection may be imminent. In this case, it makes sense to disinfect the garment, and apply a shortened skin treatment immediately.

In the winter, infection from the outside may occur once every two or three month. In the summer, it should be expected more often, around once a month. We do not know how it happens. Axillary malodor does not seem to be easy to contract, even from a domestic partner, even through a sexual intercourse. From the other hand, people may develop axillary malodor at the same time, as if it has been contracted in some place they have visited together.

There are reasons to expect, however, that chances to meet this infection will drop with increase of the percentage of the people being free from it most of the time.

If axillary odor occasionally gets stronger then usual, it's not a reason for a medication. The condition may disappear the following morning, or in a few days. To recognize the infection one may sample axillary sweat with a hand, and smell it. If the odor is pungent, and stays for several minutes, it's probably time to take care of it.

As soon as reinfection is suspected, fast and comprehensive actions must be taken to exterminate it completely, and prevent from spreading to other people. With this in mind, it usually makes sense to wait a day or two. Occasional outbursts of bad armpit odor often pass away soon without medication, especially after ample sweating. According to our observations it is also beneficial to avoid using surfactants on armpits, thus preserving natural secretions of the skin.

H. Advanced Aspects of the Invention

With regards to antimicrobial resistance issue, we would like to stress one more time that azoles are not the only mean of exterminating axillary malodors. There still are antibiotics, sulfa drugs, peroxides, halogens, etc. Besides, it's just hardly possible to get a one-time medication wrong.

Azoles may be used as occasional topical prophylactics. Having learned about our methods some people even ventured to apply them on a daily basis. Although nothing apparently bad has happened to them yet, it is not clear if such a practice is justifiable. Even being harmless to human health, it may promote antimicrobial resistance.

In public places, fomites may be taken more broadly, to include floor, carpeting, etc. Sanitizing may help to contain the spread of infections producing axillary malodors among employees and visitors.

We believe, however, that the most important aspect of the present invention is the very fact that axillary malodor may be not inevitable. It is curable, and, once it has being cured, it is not easy to contract again. We may learn to avoid axillary malodor, as we have learned to avoid many other skin infections.

### I. EXAMPLE

A woman, 31, low perspiration, axillary malodor since puberty. Successfully treated with miconazole (two applications per day, seven days in a row). Reinvested from the old shirts two times before realized that they all better be disinfected before use. Repeated infections cured with miconazole (overnight, two times in a row) or tioconazole (overnight, one-time treatment). Now uses peroxide bleach all the time. Had unusual bitter axillary odor in the summer, and slight sweaty odor as the winter came. No much need in antiperspirants or deodorants since the first treatment.

We claim:

1. A method for preventing a malodor producing in a human axilla comprising
applying to said axilla a germicidal composition comprising at least one substance selected from the group consisting of azoles, imidazoles, clotrimazole, miconazole, tioconazole, butoconazole, econazole, terconazole, fenticonazole, ketoconazole, terbinafine, tolnaftate, undecylenic acid, salicylic acid, peroxides, and derivatives thereof, in an amount effective to terminate said malodor producing,
whereby sustained deodorizing effect achieved through safe, affordable, practical, non-prescription medication.

2. A method for stopping producing of an axillary malodor in fomites, and preventing transmitting of producing of said axillary malodor from said fomites to humans comprising treating said fomites with a sanitizing agent in an amount effective to exterminate residual substances and microorganisms involved in producing of said axillary malodor.

3. The method of claim 2, wherein said sanitizing agent selected from the group consisting of azoles, imidazoles, antibiotics, derivatives thereof, oxygen, halogens, ammonium, heat, laundry detergents, and combinations thereof.

4. A method for curing a malodor producing in a human axilla comprising
applying to said axilla a dermatologically acceptable germicidal agent in an amount effective to ensure sustained alleviation of said malodor,
identifying potential fomites contacted said armpit,
treating said fomites with a sanitizing agent in an amount effective to destroy residues and microorganisms producing said malodor,
whereby protracted relief from said malodor is achieved.

5. The method of claim 4, wherein said germicidal agent selected from the group consisting of azoles, imidazoles, clotrimazole, miconazole, tioconazole, butoconazole, econazole, terconazole, fenticonazole, ketoconazole, terbinafine, tolnaftate, antibiotics, neomycin, polymyxins, gramicidin, bacitracin, sulfa drugs, halogens, undecylenic acid, salicylic acid, peroxides, derivatives thereof, and combinations thereof.

6. The method of claim 4, wherein said sanitizing agent selected from the group consisting of azoles, imidazoles, antibiotics, derivatives thereof, oxygen, halogens, ammonium, heat, laundry detergents, and combinations thereof.

7. The method of claim 6, wherein said germicidal agent selected from the group consisting of azoles, imidazoles, clotrimazole, miconazole, tioconazole, butoconazole, econazole, terconazole, fenticonazole, ketoconazole, terbinafine, tolnaftate, antibiotics, neomycin, polymyxins, gramicidin, bacitracin, sulfa drugs, halogens, undecylenic acid, salicylic acid, peroxides, derivatives thereof, and combinations thereof.
